(19) United States
(12) Patent Application Publication
(10) Pub. No.: US 2006/0094781 A1
(43) Pub. Date: May 4, 2006

(54) Method of treating extracellular matrix

(75) Inventors: Simon Eckhouse, Haifa (IL); Michael Kreindel, Haifa (IL); Joseph Shiloh, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Syneron Medical Ltd., Yokneam Illit (IL)

(21) Appl. No.: 10/980,822

(22) Filed: Nov. 4, 2004

(51) Int. Cl. A61K 31/22; A61K 31/198

(52) U.S. Cl. 514/551; 514/561

(57) ABSTRACT

A method for treating skin tissue or extracellular matrix in a body region. A precursor of a photosensitive compound is introduced into the body region. One or more enzymes converting the precursor to the photosensitive compound are then introduced into the body region so as to convert the precursor to the photosensitive compound in the extracellular matrix of the body region.

### FIELD OF THE INVENTION

This invention relates to methods for treating skin tissue and the extracellular matrix.

### BACKGROUND OF THE INVENTION

Photosensitive compounds are often used in the treatment of various forms of cancer, such as skin cancer in what is known as “photo-dynamic therapy” (PDT). In this method, a photosensitive compound is delivered into skin cells and is then exposed to light so as to produce a biologically active agent inside the cells. The biologically active agent may be, for example, singlet ¹O₂ which induces cell death. In some cases, a precursor of a photosensitive compound is introduced into cells which is converted into a photosensitive compound inside the cells by endogenous cellular enzymes.

For example, 5-Aminolevulinic acid (ALA) (also known as δ-aminolevulinic acid (δ-ALA), 5-amino-4-oxopentanoic acid, and δ-amino-γ-keto-valeric acid), is a naturally occurring compound used as a precursor of a photosensitive compound in photodynamic therapy of cancer and pre-cancer conditions such as actinic keratosis. ALA, or one of its derivatives, is applied to a skin surface to be treated. The applied ALA penetrates into the skin and is taken up by skin cells. Inside the cells, the ALA or its derivative is enzymatically converted into the photo-sensitive compounds Protoporphyrin IX (PpIX) by endogenous cellular enzymes such as δ-aminolevulinic acid dehydratase (ALAD), prophobilinogen deaminase (PBGD), uroporphyrinogen III cosythetase, uroporphyrinogen decarboxylase, coproporphyrinogen oxidase, photoporphrinogen oxidase and ferrochelatase. The amount of PpIX in the cells reaches a maximum at about 4-12 hours following application. The patient then returns to the care giver and the skin surface to be treated is exposed to blue or red light from a lamp for about 20 minutes, or exposed to a short flash from a laser or an Intense Pulse Light (IPL) source. Light at about 410 nm is used for treating superficial lesions. Longer wavelengths (at about 630 nm) are used for reaching deeper sites in the skin. The light activates the protoporphyrin and as a consequence singlet ¹O₂ is produced inside the cells which induces the death of the cells that took up the ALA or derivative.

The amount of the photosensitive compound that forms in the skin from the precursor is limited by several factors such as the activity of the enzyme or enzymes that convert the precursor into the photo-sensitive compounds in theskin cells, the concentration of the applied precursor, the time of the application, and the formulation of the applied composition. The rate of the conversion of a given amount of the precursor to the photosensitive compound is limited primarily by the endogenous levels of the cellular enzymes. As a result, a relatively high amount of precursor must be used and intense light is needed in order to obtain a sufficiently high level of the photosensitive compound inside the cells. In these treatments, the relatively high concentration of the precursor and the high intensity of the irradiation after application of the precursor may cause the subject to experience pain, irritation, and a strong erythemic reaction.

U.S. Pat. No. 5,474,528 to Meserol describes PDT treatment in which a patch with a photo-sensitizer is applied to a dermal lesion and controlled irradiation is carried out using optical energy produced by a light source.

U.S. Pat. No. 5,441,531 to Azrate et al., discloses PDT treatment in which light in the range of 600-700 nm produced by a lamp is used.

U.S. Pat. No. 5,489,279 to Meserol describes a sealed applicator for applying a photo-sensitizer such as ALA to skin for PDT treatment.

U.S. Pat. No. 5,856,566 to Golub discloses use of colored ALA where the color is imparted by irradiation of ALA crystals.

U.S. Pat. No. 5,707,401 to Talmore describes a device for simultaneous PDT treatment and hyperthermia.

U.S. Pat. No. 5,776,175 to Eckhouse et al., describes use of pulsed incoherent light for PDT treatment of tumors.

U.S. Pat. Nos. 5,422,093, 5,234,940, 5,079,262, and 5,955,490 to Kennedy et al., describes the treatment of rapidly growing skin cells by application of ALA onto skin lesions and exposing the lesions to light.

U.S. Pat. No. 5,876,989 to Berg et al., describes a method for releasing molecules into the cytosol of cells by using light activation of photosensitizing compounds to rupture the membranes of the cell compartments.

### DESCRIPTION OF THE INVENTION

The present invention provides a method for treating skin tissue and the extracellular matrix. In accordance with the invention, a precursor of a photosensitive compound is delivered to the extracellular matrix. Enzymes that convert the precursor into the photosensitive compound are also delivered to the skin tissue so as to allow the accumulation of the photosensitive compound in the extracellular matrix. The extracellular matrix is then exposed to light so as to produce a biologically active compound in the compartment. After accumulation of the photosensitive compounds in the extracellular matrix, the photosensitive compounds are irradiated with light so as to generate a biologically active agent, such as singlet oxygen, in the extracellular matrix, as explained above.

The inventors have discovered that generation of a biologically active agent in the extracellular matrix stimulates skin remodeling. Without wishing to be bound by a particular theory, it is believed that the generation of the biologically active agent in the extracellular matrix induces sensitization and metabolic stimulation of the cells of the extracellular matrix (keratocytes, fibroblasts and muscle cells). This results in enhanced release of growth factors from the cells of the extracellular matrix which leads to stimulation of the skin. As a consequence, enhanced synthesis of collagen and other extracellular matrix proteins occurs, as well as collagen shrinkage and remodeling, which induces tissue remodeling and wrinkle reduction. Under these conditions only marginal amounts of the biologically active agent are formed inside the skin cells that do not cause significant cell damage or necrotic reactions following an application of light.

In one embodiment of the invention, the enzyme or enzymes are injected into the extracellular compartment by conventional injection or using structure microneedles. In another embodiment of the invention, the delivery to the skin of the enzymes is done with a transdermal delivery device using technologies such as electrically based techniques (iontophoreses, electroporation, ultrasound, photomechanical wave) or velocity based jet propulsion.

In another embodiment, if the enzyme or enzymes that convert the precursor to the photosensitive component are present in blood cells, the permeability of the blood vessel walls in the body region may be increased so as to allow the release of the enzymes from the blood circulation into the extracellular space. In a presently preferred embodiment, the blood vessel walls in the body region are hemorrhaged, so as to create a massive infusion of blood into the extracellular matrix of the body region. For example, irradiating a skin region with an intense light source causes the blood vessels in the skin region to hemorrhage. The effusion of blood into the skin region raises the level of enzymes in the skin region.

In the case that the precursor is ALA or one of its derivatives, the inventors have found that irradiation of a skin region with light leads to an increase in the extracellular matrix of the enzymes mentioned above that convert ALA and its derivatives to PpIX, which in turn enhance the rate of conversion of the ALA to PpIX. It was also found that the increase of extracellular PpIX after the application of light is due to damage to blood vessels in the region by the light which causes effusion of blood from the vessels into the extracellular matrix and the release of ALAD and PBGD from red blood cells into the extracellular matrix. The light is preferably in the region of >600 nm, and is supplied prior to or a short time after the application of ALA.

The method of the invention may be used in the treatment of skin rejuvenation via remodeling of the extracellular matrix, acne treatment, skin bleaching and the treatment of vascular and pigmented lesions.

Non-limiting examples of ALA derivatives that may be introduced into the extracellular matrix, include lipophilic ester derivatives thereof, such as ALA hexyl ester, ALA benzyl ester, ALA pentyl ester or ALA methyl ester.

In a preferred embodiment the precursor is applied topically to the skin region. The agent may be formulated as oil, gel, ointment, paste, spray, sticks, cream or any other forms known in the art. To this end, the agent may be combined with thickening agents, gelling agents, suspension agents, emulsifiers, dispersing agents depending on the desired characteristics of the formulation. Those versed in the art of pharmacy will know how to select the specific excipients for proper formulation of the ALA. The ALA precursor may also be combined with other agents known to be used with topical formulations. The invention thus provides a method for treating skin tissue or extracellular matrix in a body region comprising:
(a) introducing a precursor of a photosensitive compound to the body region; and
(b) introducing one or more enzymes converting the precursor to the photosensitive compound in the body region so as to convert the precursor to the photosensitive compound in the extracellular matrix of the body region.

1. A method for treating skin tissue or extracellular matrix in a body region comprising:
(c) introducing a precursor of a photosensitive compound to the body region; and
(d) introducing one or more enzymes converting the precursor to the photosensitive compound in the body region so as to convert the precursor to the photosensitive compound in the extracellular matrix of the body region.

2. The method according to claim 1 further comprising irradiating the photosensitive compound in the extracellular matrix with light so as to generate a biologically active agent in the extracellular matrix.

3. The method of claim 1, wherein the precursor is 5-Aminolevulinic Acid (ALA) or a derivative thereof.

4. The method according to claim 3 wherein the ALA derivative is selected from lipophilic ester derivatives of ALA, ALA hexyl ester, ALA benzyl ester, ALA pentyl ester, and ALA methyl ester.

5. The method of claim 1, wherein the enzymes are injected into the skin.

6. The method according to claim 1 wherein introducing the enzymes into the body region comprises transdermal delivery selected from microneedle injection, iontophoreses, electroporation, ultrasound, photomechanical waves, or velocity based jet propulsion.

7. The method according to claim 1 wherein introducing the enzymes comprises damaging blood vessel walls in the body region so as to cause effusion of blood into the extracellular matrix of the body region.

8. The method according to claim 7 wherein the blood vessel walls are damaged by exposure to light.

9. The method according to claim 8 wherein the light has a wavelength greater than 600 nm.

10. The method according to claim 8 wherein the enzymes are released from blood cells.

11. The method according to claim 10 wherein the blood cells are red blood cells.

12. The method according to claim 1 wherein one or more of the enzymes are selected from delta-aminolevulinic acid dehydratase (ALAD), prophobilinogen deaminase (PBGD), uroporphyrinogen III cosythetase, uroporphyrinogen decarboxylase, coproporphyrinogen oxidase and photoporphrinogen oxidase.

13. The method according to claim 2 wherein the biologically active compound is singlet oxygen.

14. The method of claim 1, wherein the precursor is applied to the skin in the form of a cream.

15. The method according to claim 1 for use in a treatment selected from the treatment of skin rejuvenation via remodeling of the extracellular matrix, skin bleaching, the treatment of vascular and pigmented lesions, collagen shrinkage, collagen remodeling and wrinkle reduction.
